(19) United States
(12) Patent Application Publication
(10) Pub. No.: US 2026/0102110 A1
(43) Pub. Date: Apr. 16, 2026

(54) DIAGNOSTIC DEVICE AND METHOD FOR MONITORING BODY TISSUE OF A PATIENT

(71) Applicant: Roche Diagnostics Operations, Inc., Indianapolis, IN (US)

(72) Inventor: Oscar Gutiérrez-Sanz, Heidelberg (DE)

(21) Appl. No.: 19/116,264

(22) PCT Filed: Sep. 27, 2023
(86) PCT No.: PCT/EP2023/076635

§ 371 Date: Mar. 27, 2025

(30) Foreign Application Priority Data
Sep. 28, 2022 (EP) 22198423.0

(51) Int. Cl. A61B 5/00; A61B 5/107

(52) U.S. Cl. CPC A61B 5/442; A61B 5/1072; A61B 5/4878; A61B 5/681

(57) ABSTRACT

A diagnostic device (110) for monitoring at least one body tissue (134) of a patient is disclosed. The diagnostic device (110) comprises:

a. at least one bracelet (112) configured to be strapped around a body part (132) of the patient;

b. at least one electromechanical actuator (114) configured for actively varying a circumference of the bracelet (112);

c. at least one measurement unit (126) configured for determining at least one item of information on an electrical power applied to the electromechanical actuator (114) and at least one item of circumference information on the circumference of the bracelet (112); and

d. at least one evaluation unit (128) configured for determining at least one item of information on a status of the body tissue (134) from the item of information on the electrical power applied to the electromechanical actuator (114) and the item of circumference information, wherein the evaluation unit (128) is configured for determining a point of contact at which the circumference of the bracelet (112) corresponds to the circumference of the body part.

Further, a method of monitoring at least one body tissue (134) of a patient is disclosed.

### TECHNICAL FIELD

The invention relates to a diagnostic device for monitoring at least one body tissue of a patient. The invention further relates to a method of monitoring at least one body tissue of a patient, as well as to certain aspects of computer-implementation of the method. The devices and method specifically may be used for monitoring body swellings of a body tissue of a patient, such as edema or congestions, specifically ankle edema. Additionally or alternatively, other body parts may be monitored, such as body tissue of one or more a thigh, a lower thigh, a wrist, a forearm and an upper arm of the patient. The invention specifically may be applied in patient monitoring in the fields of cardiology and internal medicine. Other fields of application, however, are also feasible.

### BACKGROUND ART

In several fields of medicine, monitoring the properties of body tissue of patients is an essential part of the medical healthcare. Specifically, in the fields of cardiology and internal medicine, the examination of the properties of specific body tissues is essential, e.g. in order to detect or monitor swellings or edema. As an example, monitoring ankle edema or other congestions is an important part of cardiac healthcare, since ankle edema are known to provide an efficient evidence for the risk of heart failure symptoms. Consequently, in many fields of medicine, monitoring the properties of body tissue is required. As an example, a major challenge in the management of heart failure patients is the monitoring of congestions and body tissue, since congestions are amongst the most important determinants of heart failure symptoms and a major prognostic factor in heart failure. Congestions are mostly treated with diuretics, such as loop diuretics, as recommended e.g. by major international guidelines.

It is, therefore, of paramount importance to provide means and methods for adequately determining certain parameters of body tissues, such as the congestive status of patients. Residual congestion at discharge is associated to higher one-year mortality and heart failure readmissions. Although many clinical signs and symptoms of congestion have been well characterized and are recognized by published guidelines, no single element from clinical history or physical examination can accurately detect the underlying hemodynamic changes that lead to congestion.

A technical challenge, however, resides in the availability of robust and reliable clinical tools able to monitor and evaluate the evolution of congestions in a patient. Different manual approaches are used by professionals which, however, are highly dependent on the experience of the healthcare professional. In general, congestion evaluation tools can be divided in three groups, but so far, none of these tools alone provide sufficiently meaningful results for avoiding early discharge of patients from the hospitals or for providing suitable healthcare applicable by the patient at home. Thus, clinical tools for evaluating congestion, imaging tools and pressure and impedance tools are generally known for monitoring body tissue. Clinical tools typically comprise monitoring signs and symptoms such as dyspnea, orthopnea, or other signs and symptoms. Further, clinical tools may comprise clinical congestion scores, such as the Stevenson Score, the Everest Score, the Rhode Score, and others. Further, the clinical tools may comprise using circulating biomarkers, such as BNP, NT-proBNP, and others. Further, certain clinical measurements may be performed, including measurements of the plasma volume, hematocrit measurements, serum protein measurements, albumin measurements, and the like. Similarly, clinical tools may also comprise monitoring renal markers, such as potassium, creatinine, and others. Finally, clinical monitoring may comprise monitoring blood circulation parameters, such as central venous pressure, right atrial pressure, and the like. The second group, which is the group of using imaging tools, may comprise one or more of chest X-Ray, echocardiography, or others. Finally, the third group of pressure and impedance tools, generally, may comprise heart catheterization, the use of pressure sensors such as CardioMEMS™M Sensors, bioimpedance vector analysis (BIVA), and the like.

Further, various sensors are known in the art which have been described as useful for monitoring congestions such as ankle edema. As an example, in L. Beker: “Wearable sensors of the elasticity of deeper skin”, Nature Biomedical Engineering, vol. 5, Jul. 2021, pp. 641-642 (www.nature.com/natbiomedeng), a method of dynamically measuring the elastic modulus of the superficial and deep layers of the skin via wearable conformal electromagnetic devices consisting of a vibratory actuator and a soft strain-sensing sheet is described.

Similarly, in R. Fallahzadeh et al.: “Smart-Cuff: A Wearable Bio-Sensing Platform with Activity-Sensitive Information Quality Assessment for Monitoring Ankle Edema”, conference paper, The 7th International Workshop on Information Quality and Quality of Service for Pervasive Computing (IQ2S) in Conjunction with IEEE PerCom, March 2015 (https://www.researchgate.net/publication/307958193_Smart-Cuff_A_Wearable_Bio-Sensing_Platform _with_Activity-Sensitive_Information_Quality_Assessment_for_Monitoring_Ankle_Edema), a wearable real-time platform is disclosed, which integrates advanced technologies in sensing, computation, and signal processing and machine learning for continuous and real-time edema monitoring in remote and in-home settings.

In U.S. Pat. No. 8,827,930 B2, systems and methods of monitoring a patient are disclosed. Exemplary methods include receiving sensor data associated with the patient from a plurality of sensors of a patient monitoring device and determining whether the sensor data satisfies one or more trigger conditions. For each of the trigger conditions satisfied, one or more messages are sent to at least one of the patient monitoring device and an external computing device for display thereby to at least one of the patient, a caregiver, and a support person. Satisfaction of one or more of the trigger conditions may indicate the patient has edema and/or is trending toward decompensation. The sensor data may have been collected from a heart rate sensor, an oximeter, an accelerometer, and/or a sensor configured to detect a distance around a limb of a patient. In some embodiments, the trigger conditions are provided by the patient, caregiver, and/or support person.

U.S. Pat. No. 10,206,621 B2 discloses devices, system, and a method for the prediction and prevention of acute decompensated heart failure or other patient conditions involving fluid accumulation in legs or hands. In one example, a wearable device contains a drift-free leg-size sensor and a tissue-elasticity sensor. Both sensors may be relatively inexpensive and developed using innovative new sensing ideas. Preliminary tests with the sensor prototypes show promising results: the leg-size sensor is capable of measuring 1 mm changes in leg diameter and the tissue-elasticity sensor can detect 0.15 MPa differences in elasticity. In another example, a wearable system includes sensors for measuring a variety of physiological parameters, a processing module, and a communication module. A low-profile instrumented sock, e.g., a wearable device, with multiple sensors can provide an indication of heart failure status for a patient.

US 202110015426 A1 discloses a measurement apparatus capable of measuring the amount of edema of an ankle. The measurement apparatus includes a sensor that has a sheet shape and freely expands and contracts in one direction that intersects a thickness direction. The measurement apparatus is capable of detecting change in an electrical characteristic in association with expansion or contraction. The measurement apparatus further includes a support member that is attached to both end portions and in the expansion-contraction direction in the sensor and surrounds the ankle together with the sensor in a mounted state in which the measurement apparatus is mounted on the ankle.

US 2010/0010406 A1 discloses a self-contained compression device and related method for cyclically compressing the limb of a patient to improve blood flow in the limb. In one embodiment, the compression device includes a compressive section sized and shaped for extending around a portion of the limb for applying compressive pressure and a housing operatively connected to the compressive section. The housing includes first and second housing members movable relative to each other between contracted and expanded positions. A nonpneumatic mechanical actuator is provided in the housing for cyclically moving the first and second housing members from their contracted position to their expanded position. In one embodiment, the actuator comprises a prime mover and at least one cam movable by the prime mover for effecting relative movement between the first and second housing members.

CN 213 345 640 U discloses a breast cancer postoperative lymphedema electric measuring device which comprises a tape, a plurality of gear grooves arranged in the middle of the tape, scale marks arranged on the surface of the tape, wherein the scale marks take one side of the tape as a starting point, and the other side of the tape as a terminal point. A measuring installation box is fixedly installed on the right side surface of the tape, a fixed installation block is fixedly installed in the measuring installation box, an installation groove is formed in the fixed installation block, a gear is installed in the installation groove, a rotating shaft is installed on the gear, one end of the rotating shaft is in transmission connection with the output end of a motor through a coupler, and the other end of the rotating shaft is in transmission connection with the output end of the motor. The gear groove formed in the tape is meshed with the gear fixedly installed in the measurement installation box, the motor drives the gear to rotate, and therefore the tape can be automatically tightened through the gear for measurement, and measurement data can be conveniently observed through the scale marks.

JP 2008 096315 A discloses a means for winding a belt around an object for which elasticity is to be evaluated that is constructed to represent pulling the end of the wound belt, measuring the tensile force generated in the belt, and measuring the amount of change in the length of the portion where the belt is wound around the measurement object.

Despite the advantages achieved with these technical devices and methods, several technical challenges remain. Thus, there still is a need for robust and reliable tools to be used either in a clinical environment or at home, by healthcare professionals or by untrained persons such as the patient himself or herself. Specifically, means and methods that are widely independent on an environmental conditions like temperature are at need and which do not require for sophisticated in situ calibration.

### Problem to Be Solved

It is therefore desirable to provide devices and methods which, at least partially, address the above-mentioned technical challenges. Specifically, devices and methods for monitoring body tissue shall be proposed which are robust and provide for reliable results indicating a state of the body tissue.

### SUMMARY

This problem is addressed by a diagnostic device for monitoring at least one body tissue of a patient, as well as by a method of monitoring at least one body tissue of a patient and certain aspects of computer-implementation of the method, with the features of the independent claims. Advantageous embodiments which might be realized in an isolated fashion or in any arbitrary combination are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a diagnostic device for monitoring at least one body tissue of a patient is disclosed. The diagnostic device specifically may be used for monitoring body swellings of a body tissue of the patient. The diagnostic device comprises:
a. at least one bracelet configured to be strapped around a body part of the patient, specifically around one or more of an ankle, a thigh, a lower thigh, a wrist, a forearm and an upper arm of the patient;
b. at least one electromechanical actuator configured for actively varying a circumference of the bracelet;
c. at least one measurement unit configured for determining at least one item of information on an electrical power applied to the electromechanical actuator and at least one item of circumference information on the circumference of the bracelet; and
d. at least one evaluation unit configured for determining at least one item of information on a status of the body tissue from the item of information on the electrical power applied to the electromechanical actuator and the item of circumference information, particularly wherein the evaluation unit is configured for determining a point of contact at which the circumference of the bracelet corresponds to the circumference of the body part.

The term “diagnostic device” as used herein is a broad term and is to be given its ordinary and customary meaning to a person of ordinary skill in the art and is not to be limited to a special or customized meaning. The term specifically may refer, without limitation, to an arbitrary device or a combination of devices which are capable of determining at least one parameter indicative of a state of a human or animal being, such as at least one physiological or medical parameter.

Similarly, the term “monitoring” as used herein is a broad term and is to be given its ordinary and customary meaning to a person of ordinary skill in the art and is not to be limited to a special or customized meaning. The term specifically may refer, without limitation, to a process including one or more of measuring, recording and indicating at least one parameter, such as by electrical means. The result of the monitoring, as an example, may be or may comprise at least one item of information, such as at least one item of information on the electrical power applied to the electromechanical actuator, e.g. at least one analogue and/or at least one digital signal.

The term “bracelet” as used herein is a broad term and is to be given its ordinary and customary meaning to a person of ordinary skill in the art and is not to be limited to a special or customized meaning. The term specifically may refer, without limitation, to an arbitrary device or a combination of devices having at least partially deformable or flexible properties and being configured for being strapped around at least one part of the human body. The bracelet, as an example, may comprise a deformable element which may be bent around the body part, in order to form at least one loop through which the body part may extend. As an example, the bracelet may fully or partially be made of a deformable material, such as at least one of: a plastic material; a metallic material, specifically a sheet-metallic material; a textile material; a paper or cardboard material or any combination thereof.

The term “electromechanical actuator” as used herein is a broad term and is to be given its ordinary and customary meaning to a person of ordinary skill in the art and is not to be limited to a special or customized meaning. The term specifically may refer, without limitation, to an arbitrary device or a combination of devices capable of transforming at least one electrical signal or electrical energy into at least one mechanical action, such as into at least one motion, specifically into at least one of a linear motion and a rotation. Many types of electromechanical actuators are known to the skilled person and may also be used in the present invention. Specifically, the electromechanical actuator may comprise at least one electrical motor, such as an electrical motor selected from the group consisting of: a DC motor; a stepper motor.

The electrical motor specifically may be configured for actively varying the circumference of the bracelet, e.g. by widening or narrowing a loop formed by the bracelet. For this purpose, the electrical motor specifically may be configured for moving at least one first portion of the bracelet relative to at least one second portion of the bracelet, in order to vary the circumference of the bracelet. Thereby, as an example, the loop may be widened or narrowed in a controlled fashion by the electromechanical actuator, particularly an electrical motor.

The electromechanical actuator may fully or partially be separate from the bracelet or may, alternatively, fully or partially be integrated into the bracelet or attached to the bracelet. More specifically, the electromechanical actuator may be attached to or in contact with the bracelet in at least two portions of the bracelet. More specifically, the electromechanical actuator may be configured for moving one portion of the bracelet with respect to another portion of the bracelet, in order to narrow and/or widen a loop formed by the bracelet.

The term “actively” as used herein and as specifically used in the context of the varying of the circumference of the bracelet, is a broad term and is to be given its ordinary and customary meaning to a person of ordinary skill in the art and is not to be limited to a special or customized meaning. The term specifically may refer, without limitation, to the property of a process of being one or more of controlled, initiated and stopped by external influences. Thus, as opposed to e.g. varying a circumference of the bracelet by internal temperature change is within the bracelet, the active varying of the circumference may include exerting external forces and/or using external energy or energy transformations from electrical energy into mechanical energy for varying the circumference. Specifically, the process of actively varying the circumference of the bracelet may include a controlling of the varying of the circumference, e.g. by one or more control signals generated internally or externally, e.g. by a control unit of the diagnostic device. The control unit may be part of the bracelet, of the electromechanical actuator or may be located externally.

The term “measurement unit” as used herein is a broad term and is to be given its ordinary and customary meaning to a person of ordinary skill in the art and is not to be limited to a special or customized meaning. The term specifically may refer, without limitation, to a device or a combination of devices configured for measuring at least one property of a system, an element or a device. The measurement unit, as will be outlined in further detail below, may comprise a single measurement device or a plurality of measurement devices. The measurement unit specifically may be configured for generating at least one electrical measurement signal, specifically at least one of an analogue signal and a digital signal. The measurement unit may fully or partially be embodied as a separate unit or may, alternatively, also be fully or partially integrated into one or more other components of the diagnostic device, such as into at least one of: the electromechanical actuator, a control unit of the diagnostic device, the evaluation unit.

The term “item of information on an electrical power applied to the electromechanical actuator” as used herein is a broad term and is to be given its ordinary and customary meaning to a person of ordinary skill in the art and is not to be limited to a special or customized meaning. The term specifically may refer, without limitation, to an arbitrary item of information which qualifies and/or quantifies an electrical power applied to the electromechanical actuator, e.g. by an external electrical power source and/or by an internal electrical power source integrated into the diagnostic device, e.g. an accumulator and/or a battery. The term specifically may further refer, without limitation, to an arbitrary item of information that qualifies and/or quantifies an electrical power applied to the electromechanical actuator that is needed to move the electromechanical actuator by a defined distance or angle. By moving the electromechanical actuator by a defined distance or angle, the circumference of the bracelet, particularly the circumference of the loop formed by the bracelet, may be changed, either increased or decreased. This change of the circumference of the bracelet, particularly the change of the circumference of the loop formed by the bracelet, may be proportional to the movement of the electromechanical actuator by a defined distance or angle. Particularly, the circumference of the bracelet may be decreased from a first circumference to a second circumference. Thus, the item of information on the electrical power applied to the electromechanical actuator may comprise one or more of an electrical current, an electrical voltage and an electrical power. The item of information on the electrical power applied to the electromechanical actuator specifically may be provided by the measurement unit in an electrical format, such as in the form of an analogue and/or digital electrical signal.

The term “circumference” as used herein is a broad term and is to be given its ordinary and customary meaning to a person of ordinary skill in the art and is not to be limited to a special or customized meaning. The term specifically may refer, without limitation, to a parameter indicating a length of a loop formed by the bracelet, through which the body part of the patient may extend. The circumference may be an inner circumference, particularly of an inner side of the bracelet that may be configured for being in contact with the body part of the patient when the bracelet is strapped around the body part of the patient. Thus, as an example, a diameter of the loop formed by the bracelet may be proportional to the circumference of the loop and may particularly be or may be determined by using the circumference information. The diameter may be an actual diameter of a round loop, or may be related to other parameters indicating the width of the loop, e.g. an equivalent diameter.

The term “item of circumference information” or “item of information on the circumference of the bracelet” as used herein is a broad term and is to be given its ordinary and customary meaning to a person of ordinary skill in the art and is not to be limited to a special or customized meaning. The term specifically may refer, without limitation, to an arbitrary item of information which qualifies and/or quantifies a current circumference, a current diameter or equivalent diameter of the bracelet, particularly of a loop formed by the bracelet, including the option of providing information on actual values allowing for an identification of the circumference and the option of providing information on temporal derivatives thereof, such as a rate of change of the circumference, the diameter or the equivalent diameter. The circumference information may comprise an item of information on the inner circumference. This item of information may be the inner circumference itself or an arbitrary value that may be examined to conclude the inner circumference. Thus, as an example, the circumference may comprise information that is proportional to an actual diameter of a round loop, or may also comprise information on other parameters indicating the width of the loop, e.g. an equivalent diameter. As an example, the item of circumference information on the circumference of the bracelet may also comprise information on an absolute position of at least one portion of the bracelet or on a relative position of two portions of the bracelet with respect to each other and/or of at least one portion of the bracelet with respect to a reference point or reference region, such as with respect to the electromechanical actuator. The item of circumference information specifically may be provided by the measurement unit in an electrical format, such as in the form of an analogue and/or digital electrical signal, e.g. as a position signal and/or as an angular signal.

The term “evaluation unit” as used herein is a broad term and is to be given its ordinary and customary meaning to a person of ordinary skill in the art and is not to be limited to a special or customized meaning. The term specifically may refer, without limitation, to an arbitrary device or combination of devices configured for analyzing and/or processing data. The evaluation unit may specifically analyze and/or process measurement data, e.g. the measurement results as generated by the measurement unit. The evaluation unit may in particular comprise at least one processor. The processor may specifically be configured, such as by software programming, for performing one or more evaluation operations on the item of information on the electrical power applied to the electromechanical actuator and the item of circumference information.

Similar to the measurement unit, the evaluation unit also may be embodied as a separate unit or may be fully or partially integrated into one or more other parts of the diagnostic device. Thus, as an example, the evaluation unit may fully or partially be integrated into one or more of: the electromechanical actuator, a control unit of the diagnostic device, the measurement unit. Other options, however, are feasible.

The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for performing one or more evaluation operations.

The term “item of information on a status of the body tissue” as used herein is a broad term and is to be given its ordinary and customary meaning to a person of ordinary skill in the art and is not to be limited to a special or customized meaning. The term specifically may refer, without limitation, to an arbitrary item of information allowing for qualifying and/or quantifying the status of the body tissue. As an example, the item of information on the status of the body tissue may be provided, by the evaluation unit, in the form of at least one electrical signal, such as at least one analogue signal and/or at least one digital signal. The item of information on the status of the body tissue specifically may comprise at least one item of information selected from the group consisting of: a degree of swelling of the body tissue, specifically a circumference of the body tissue; and an elasticity parameter of the body tissue, specifically an elasticity module of the body tissue.

The item of information on a status of the body tissue may comprise an item of body part circumference information of the body part. The item of circumference information of the bracelet at the point of contact, as an example, may be used as the item of body part circumference information of the body tissue, or may be used for determining this item of body part circumference information. The term “point of contact” as used herein is a broad term and is to be given its ordinary and customary meaning to a person of ordinary skill in the art and is not to be limited to a special or customized meaning. The term specifically may refer, without limitation, to the bracelet being, particularly fully, attached to the body part in a manner that the circumference of the body part may be determined. At the point of contact, the circumference of the bracelet, particularly of the loop, may correspond and/or equal the circumference of the body part. The point of contact may be determined by detecting the onset of a rise in electrical power required to be applied to the actuator, e.g. by monitoring the slope of the curve indicating the electrical power as a function of the circumference information; or vice versa.

By considering the point of contact the circumference of the body part may be determined, particularly the absolute value of the circumference of the body part may be determined. The circumference of the body part may be determined in an uncompressed state of the body part, particularly at which the bracelet does not exert force on the body part, particularly at which the bracelet does not exert a further compressive force on the body part, in addition to the already applied force caused by the weight of the bracelet.

For determining the at least one item of information on the status of the body tissue from the item of information on the electrical power applied to the electromechanical actuator and the item of circumference information, the evaluation unit may be configured for using at least one transformation process, specifically at least one programmed transformation algorithm, such as at least one conversion function and/or at least one lookup table. Thereby, as an example, from the electrical power necessary for narrowing the circumference of the bracelet, against the resistance of the body tissue, an elasticity parameter of the body tissue may be derived, e.g. by using an empirical or semi-empirical transformation algorithm. As an example, the diagnostic device may be calibrated, e.g. factory calibrated, by using artificial tissues or dummies having known properties, e.g. known elastic properties, and the electrical power may be measured which is required for changing the circumference of the bracelet against the resilience of the dummies or artificial body tissues. Thereby, a calibration function or calibration data such as data for generating a lookup table, may be generated and may be used in later measurements. Alternatively, however, the raw data may be used for determining the item of information on the status of the body tissue, e.g. the raw data of the electrical power required for changing the circumference of the bracelet from a first circumference to a second circumference.

The diagnostic device specifically may comprise at least one control unit configured for controlling at least one measurement routine of the diagnostic device. The term “control unit” as used herein is a broad term and is to be given its ordinary and customary meaning to a person of ordinary skill in the art and is not to be limited to a special or customized meaning. The term specifically may refer, without limitation, to an arbitrary device or combination of devices configured for controlling one or more other devices, specifically for electrically controlling one or more other devices, by providing one or more control signals, such as electrical control signals. The control unit may comprise at least one processor. The processor may specifically be configured, such as by software programming, for controlling one or more measurement routines of the diagnostic device.

Similar to the measurement unit and the evaluation unit, the control unit may be embodied as a separate unit or may be fully or partially integrated into one or more other parts of the diagnostic device. Thus, as an example, the control unit may fully or partially be integrated into one or more of: the electromechanical actuator, the evaluation unit of the diagnostic device, the measurement unit of the diagnostic device. Other options, however, are feasible.

The term “measurement routine” as used herein is a broad term and is to be given its ordinary and customary meaning to a person of ordinary skill in the art and is not to be limited to a special or customized meaning. The term specifically may refer, without limitation, to a one or more steps, such as a sequence of measurement steps, which are can be used for acquiring the at least one item of information on the status of the body tissue. The one or more steps of the measurement routine may comprise at least one of: a step of controlling the electro-mechanical actuator; a step of measurement for determining the at least one item of information on the electrical power applied to the electromechanical actuator; a step of measurement for determining the at least one item of circumference information on the circumference of the bracelet. The steps may also or may also fully or partially be combined. Examples of measurement routines are given in further detail below.

Thus, as an example, the measurement routine comprises at least one step of elasticity measurement. The step of elasticity measurement specifically may comprise the following steps, which may be performed in sequence, specifically in the given order, which, however, may also be performed in a timely overlapping fashion or in parallel, and, in which, one or more of the steps may also be performed once or repeatedly:
adjusting the circumference of the bracelet to at least one first circumference,
varying the circumference of the bracelet, by using the electromechanical actuator, from the first circumference to at least one second circumference, specifically to at least one second circumference being smaller than the first circumference, wherein the item of information on the electrical power applied to the electromechanical actuator and the item of circumference information are recorded at least at the beginning and the end of this circumference variation process, and specifically also during this circumference variation process.

Specifically, from the recording of the information on the electrical power applied to the electromechanical actuator as a function of the circumference information, valuable information on the elasticity may be derived.

The term “elasticity” as used herein is a broad term and is to be given its ordinary and customary meaning to a person of ordinary skill in the art and is not to be limited to a special or customized meaning. The term specifically may refer, without limitation, to the deformability of an object and/or to the ability of an object to resist a distorting influence or force. After the distorting influence is removed, the object may fully or partially returned to its original size and shape. Thus, specifically, at least one item of elasticity information may be derived which quantifies or at least qualifies the defamation of the object as a function of the force applied. The object may be a body. Therein, as an example, one or more elasticity modules as known to the skilled person may be used. Additionally or alternatively, however, other types of elasticity information may also be applied in the context of the present invention.

Thus, specifically, the item of information on the status of the body tissue which is determined by the evaluation unit from the item of information on the electrical power applied to the electromechanical actuator and the item of circumference information may comprise at least one item of elasticity information on the body tissue. The evaluation unit may specifically be configured for deriving the item of elasticity information from the item of information on the electrical power applied to the electromechanical actuator and the item of circumference information recorded during the step of elasticity measurement. Thus, as an example, the item of elasticity information may be derived from one or more of: a quotient of the electrical power and a change in circumference of the bracelet in a predetermined or determinable measurement range; a quotient of a change in electrical power and a change in circumference of the bracelet in a predetermined or determinable measurement range; a slope of a curve indicating the electrical power as a function of the circumference of the bracelet or a variable correlating to the same; a slope of a curve indicating the circumference of the bracelet or a variable correlating to the same as a function of the electrical power or a variable correlating to the same. It shall be noted, however, that other types of transformations or other ways of deriving the elasticity information from the step of elasticity measurement are feasible. For determining the at least one item of elasticity information, at least one transformation may be used by the evaluation unit, such as at least one transformation function and/or at least one lookup table. Again, as an example, the transformation may be determined by one or more calibration measurements, e.g. by calibration measurements on one or more dummies and/or on one or more dummies having known elastic properties, thereby, as an example, deriving a calibration curve or a plurality of calibration points for generating a lookup table, the calibration curve or calibration points indicating the item of elasticity information as a function of the item of information on the electrical power applied to the electromechanical actuator and the item of circumference information recorded during the step of elasticity measurement.

Thus, as outlined above, the evaluation unit specifically may be configured for deriving the item of elasticity information from at least one of: a slope of a measurement curve indicating the electrical power applied to the electromechanical actuator and the circumference of the bracelet; a slope of a measurement curve indicating the circumference of the bracelet and the electrical power applied to the electromechanical actuator; at least two points of measurement, each point of measurement indicating the circumference of the bracelet and the electrical power applied to the electromechanical actuator. Other ways of deriving the item of elasticity information, however, may be used additionally or alternatively.

The above-mentioned measurement routine, in addition or as an alternative to the at least one step of elasticity measurement, may also comprise at least one step of body part circumference measurement. The step of body part circumference measurement specifically may comprise the following step, wherein the step may be performed once or repeatedly:
decreasing the circumference of the bracelet, by using the electromechanical actuator, wherein the item of information on the electrical power applied to the electromechanical actuator and the item of circumference information are recorded.

The term “body part circumference” as used herein is a broad term and is to be given its ordinary and customary meaning to a person of ordinary skill in the art and is not to be limited to a special or customized meaning. The term specifically may refer, without limitation, to a closed length, specifically a closed outer length, of the shape and/or the size of the body part around which the bracelet is strapped. Specifically, the body part circumference may be quantified by at least one item of body part circumference information. The at least one item of body part circumference information may provide an arbitrary quantification of one or both of the size and the shape of the body part. As an example, the item of body part circumference information may provide information on a circumference of the body part or, thereby may relate to a circumference, a diameter and/or an equivalent diameter of the body part.

Thus, generally, the item of information on the status of the body tissue may comprise at least one item of body part circumference information on the body tissue, e.g. in addition or as an alternative to the at least one item of elasticity information. The evaluation unit may be configured for deriving the item of body part circumference information from the item of information on the electrical power applied to the electromechanical actuator and the item of circumference information recorded during the step of body part circumference measurement. Again, for this determination of the at least one item of body part circumference information, at least one transformation may be used, such as at least one empirical or semi-empirical transformation determined by one or more calibration measurements. Additionally or alternatively, analytical solutions may be used. Thus, as an example, the evaluation unit may be configured for determining the item of body part circumference information on the body tissue from at least one of: a slope of a measurement curve indicating the electrical power applied to the electromechanical actuator and the circumference of the bracelet; a slope of a measurement curve indicating the circumference of the bracelet and the electrical power applied to the electromechanical actuator; at least two points of measurement, each point of measurement indicating the circumference of the bracelet and the electrical power applied to the electromechanical actuator. As an example, the control unit may be configured for controlling the electromechanical actuator to reduce the circumference of the bracelet, e.g. gradually or in a stepwise fashion. When the bracelet is wound loosely around the body part, initially, the electrical power required to be applied to the electromechanical actuator for this reduction of circumference is relatively low and more or less constant. Once the circumference of the bracelet, however, fits with the circumference of the body part, the body part starts exerting counter forces and/or starts resisting the distorting influence by the bracelet. Consequently, the electrical power required to be applied to the electromechanical actuator for a further reduction of circumference starts raising. By monitoring, as an example, the onset of this rise, e.g. by monitoring the slope of the curve indicating the electrical power as a function of the circumference information, the point of contact, at which the circumference of the bracelet corresponds to the circumference of the body part, may be determined. The item of circumference information of the bracelet at this point of contact, as an example, may be used as the item of body part circumference information on the body tissue, or may be used for determining this item of body part circumference information. Thus, generally, the at least one item of body part circumference information may comprise at least one item of information indicating a circumference of the body part in a rest state at which the bracelet does not exert any force on the body tissue. The circumference may be proportional to an equivalent diameter of the body part in a rest state at which the bracelet does not exert any force on the body tissue. An absolute value of the body part circumference may be determined.

The at least one step of elasticity measurement and the at least one step of body part circumference measurement may also be combined, e.g. in one and the same measurement routine. Thus, as an example, the control unit may be configured for decreasing the circumference of the bracelet by providing corresponding control signals to the electromechanical actuator. From the onset of the above-mentioned rise in the electrical power required to be applied to the actuator when the bracelet, which was originally loosely wound around the body part, gets in contact with the body tissue, firstly, the at least one item of body part circumference information may be derived by the evaluation unit. When the circumference is further reduced, the at least one item of elasticity information may be derived from the rise in electrical power required to be applied to the actuator when the circumference of the bracelet is further reduced against the resilience of the body tissue, e.g. from the slope of curve of the electrical power as a function of the circumference of the bracelet.

Additionally or alternatively, an absolute value of the circumference of the bracelet may be determined during the step of body part circumference measurement. Thereby, an absolute value of the current circumference of the respective body part on which the bracelet may be wound or strapped around may be determined. The term “length of the bracelet” as used herein is a broad term and is to be given its ordinary and customary meaning to a person of ordinary skill in the art and is not to be limited to a special or customized meaning. The term specifically may refer, without limitation, to a length of a band that is used for forming the bracelet, particularly for forming the loop of the bracelet. The loop may be formed by inserting a free end of the band into the electromechanical actuator so that the free end or loose end protrudes from the electromechanical actuator. The term “length of a loose end” as used herein is a broad term and is to be given its ordinary and customary meaning to a person of ordinary skill in the art and is not to be limited to a special or customized meaning. The term specifically may refer, without limitation, to a length of a free end of the band being used for forming the bracelet or the bracelet. The free end or loose end may, particularly, not be forming the loop of the bracelet. As an example, the free end or loose end may protrude from the loop of the bracelet. Thereby, the free end or the loose end may stand out from the electromechanical actuator, particularly when the loop of the bracelet is formed by inserting the free end of the band into the electromechanical actuator.

The absolute value of the circumference of the bracelet, particularly of a loop formed by the bracelet, may be determined by at least one of: considering an absolute length of the bracelet and an absolute length of a loose end of the bracelet; particularly wherein the absolute length of the bracelet is known and the absolute length of the loose end may be determined by using a length sensor, more particularly wherein the length sensor may be the electromechanical actuator; or considering at least one position marker on the bracelet. In case the absolute length of the bracelet and the absolute length of a loose end of the bracelet may be considered for determining the absolute value of the circumference of the bracelet, particularly of a loop formed by the bracelet, the absolute value of the circumference may be determined by subtracting the absolute length of the loose end from the absolute length of the bracelet. Additionally, a known absolute offset length value may further be considered.

The absolute length of the bracelet may be predetermined and thereby known to the evaluation unit, particularly as the absolute length of the bracelet may be fixed. The length of the loose end may vary, particularly when the bracelet is strapped around the body part to form a loop. Before the body part circumference measurement may be performed, the bracelet may be in an initial configuration in which the circumference on the loop of the bracelet may have an initial absolute value and the length of the free end may have an initial absolute value. The absolute length of the loose end may be determined by using a length sensor, particularly the absolute length of the loose end in the initial configuration may be determined. As described, the sensor may be the electromechanical actuator itself. Alternatively, the length sensor may be a device that is different from the electromechanical actuator. As an example, the length sensor, particularly the electromechanical actuator, may be used to determine the number of teeth of the toothed rack that are inserted into the electromechanical actuator, particularly for forming the loop of the bracelet by the patient. Further, the number of the teeth may be determined, that is involved is actively changing the circumference of the bracelet by the electromechanical actuator. Alternatively or in addition, the absolute value of the circumference may be determined by considering at least one or a plurality of position markers on the bracelet. For considering the at least one position marker the length sensor may be used. Alternatively or in addition, the patient and/or a further person may be asked to indicate at least one value related to the position marker that is currently to be considered.

As outlined above, as an example, the electromechanical actuator specifically may comprise at least one electrical motor. Generally, as an example, the electromechanical actuator specifically may be configured for performing a rotational movement, e.g. for narrowing or widening a loop formed by the bracelet. As an example, the bracelet may comprise at least one toothed belt or rack configured for interacting with the electromechanical actuator. The diagnostic device may comprise at least one pinion driven by the electromechanical actuator, the pinion being configured for interacting with the toothed belt or rack. Thereby, as an example, the circumference of the bracelet may be reduced by rotating the electromechanical actuator in one direction and may be increased by rotating the electromechanical actuator in an opposite direction.

As outlined above, the diagnostic device comprises at least one measurement unit. The measurement unit may comprise at least one electrical measurement device configured for determining the at least one item of information on the electrical power applied to the electromechanical actuator and at least one position measurement device configured for determining the at least one item of circumference information on the circumference of the bracelet, particularly of a loop formed by the bracelet. As an example, the electrical measurement device may comprise at least one of a voltage measurement device and a current measurement device. Thereby, an electrical power applied to the electromechanical actuator may be measured. As outlined above, the measurement unit may also fully or partially be combined with the at least one optional control unit, e.g. by fully or partially being integrated into the same, thereby directly having access to the electrical power applied to the electromechanical actuator. Other ways, however, for determining the electrical power are also feasible.

The position measurement device specifically may be configured for determining at least one of: an absolute position of at least one part of the bracelet; a relative position of at least one first portion of the bracelet relative to at least one second portion of the bracelet; a temporal change of an absolute position of at least one part of the bracelet; a change in a relative position of at least one first portion of the bracelet relative to at least one second portion of the bracelet. For this purpose, various types of position measurement devices may be used, alone or in combination. As an example, when using the above-mentioned rotational electromechanical actuator and/or the pinion and/or the toothed belt or rack, a linear position and/or a rotational position may be measured, specifically of one portion of the bracelet with respect to another portion. As an example, generally, the electromechanical actuator may be fixed to one portion, preferably to a first position, of the bracelet and may be configured for moving another portion of the bracelet with respect to the first portion. By measuring a relative position of the first portion and the second portion, the position measurement may be performed, allowing for deriving information on the circumference of the bracelet, particularly of a loop formed by the bracelet. As an example, the position measurement device may comprise at least one rotational status of the electromechanical actuator and/or of the at least one pinion may be measured. In case the electromechanical actuator comprises at least one stepper motor, the item of circumference information may directly or indirectly be derived from at least one signal provided by the stepper motor. Additionally or alternatively, at least one sensor for detecting a rotational state of the motor and/or the pinion may be used, such as a magnetic sensor and/or an optical sensor. Additionally or alternatively, at least one linear position sensor may be used, in order to, as an example, determine a position of the further portion of the bracelet with respect to the electromechanical actuator and/or the first portion. Again, as an example, optical sensors may be used. Various types of others sensors are feasible, as the skilled person will recognize.

In a further aspect of the present invention, a method of monitoring at least one body tissue of a patient is disclosed. The method comprises the following steps. The steps specifically may be performed in the given order. A different order, however, is also feasible. Further, two or more of the method steps may be performed in a timely overlapping fashion or in parallel. Further, one or more or even all of the method steps may be performed repeatedly.

The method comprises the following steps:
i. providing at least one diagnostic device according to the present invention, e.g. according to any one of the embodiments described above and/or according to any one of the embodiments described in further detail below;
ii. strapping the bracelet around a body part of the patient;
iii. actively varying the circumference of the bracelet by using the electromechanical actuator;
iv. determining at least one item of information on an electrical power applied to the electromechanical actuator and at least one item of circumference information on the circumference of the bracelet by using the measurement unit; and
v. determining at least one item of information on a status of the body tissue from the item of information on the electrical power applied to the electromechanical actuator and the item of circumference information by using the at least one evaluation unit, particularly and determining a point of contact at which the circumference of the bracelet corresponds to the circumference of the body part by using the at least one evaluation unit.

As outlined above, one or more or even all of the method steps may be performed repeatedly. Thus, as an example, at least method steps iii. to v. may be performed repeatedly, e.g. by forming a routine, wherein these method steps, as an example, are repeated in regular or irregular time intervals and/or whenever triggered by a user or the patient. The routine of measurement may be controlled, as an example, by the control unit.

The method may also fully or partially be computer-implemented and/or computer control. Thus, as an example, at least step v. is computer-implemented, e.g. by a corresponding computer program running on the evaluation unit, e.g. the processor of the evaluation unit. Further, as outlined above, one or more measurement routines may be controlled by the at least one control unit. Thus, additionally or alternatively, as an example, steps iii. and/or iv. may fully or partially be computer-controlled, e.g. by a computer program running on the control unit, e.g. a processor of the control unit.

Consequently, in a further aspect, a computer program is disclosed, comprising instructions which, when the program is executed by the evaluation unit of the diagnostic device according to the present invention, e.g. according to any one of the embodiments described above and/or according to any one of the embodiments described in further detail below, cause the evaluation unit to perform step v. of the method according to the present invention, e.g. according to any one of the embodiments described above and/or according to any one of the embodiments described in further detail below.

Similarly, in a further aspect, a computer-readable storage medium, specifically a non-volatile computer-readable storage medium, is disclosed, comprising instructions which, when the instructions are executed by the evaluation unit of the diagnostic device according to the present invention, e.g. according to any one of the embodiments described above and/or according to any one of the embodiments described in further detail below, cause the evaluation unit to perform step v. of the method according to the present invention, e.g. according to any one of the embodiments described above and/or according to any one of the embodiments described in further detail below.

The invention according to any one of the embodiments described above or according to any one of the embodiments described in further detail below provides a large number of advantages over known means and methods, specifically over the above-mentioned known methods and devices. Thus, specifically, the diagnostic device and the method may provide for sensing means and methods to quantify at least the swelling of the ankle and/or wrist of the patient by means of the bracelet, which, as an example, may generally be designed as a band such as a wristband and/or an ankle band. Thereby, as an example, a circumference of the ankle and/or the wrist and/or an elasticity of the body tissue at the ankle and/or the wrist may be determined.

The diagnostic device advantageously provides a simple, durable and cost-efficient setup, particularly as no further sensor measuring the tensile force produced in the belt is required. Typically, such a further sensor may be a gauge or a load cell having a short durability. According to the invention, the information required for determining the item of information on a status of the body tissue is derived from a parameter associated with the electromechanical actuator. Thereby, the precision of the measurement increases significantly as no further sensor being associated with measurement errors is required and, typically, parameters of an electromechanical actuator, such as an electrical power applied to the electromechanical actuator, may be determined in a highly precise and accurate fashion. Further, as the electromechanical actuator is comprised by a fastener designed forming a loop of the bracelet, the diagnostic device is particularly compact.

The results provided by the diagnostic device and/or the method as proposed herein specifically may be combined with additional sensor signals, patient's parameters or vital signs, like skin-impedance or the like. At least one additional sensor signal may be selected from the group consisting of: a temperature signal, an electrocardiogramal, a pulse signal, a partial oxygen pressure (pO₂) signal, a motion signal, particularly generated by an accelerometer, a breathing signal, a pulmonary artery (PA) pressure signal, a body weight signal. Thereby, a combination of various sensor signals, patient's parameters or vital signs may be used for diagnosis. Evaluation routines such as self-learning models may be applied to data provided by the diagnostic device and optionally other devices, e.g. in order to predict a risk of heart failure and other diseases for the patient.

The diagnostic device specifically may be designed as a wearable device, which may be worn by the patient at home and/or in hospital care settings. The option of quantification of an ankle swelling and/or wrist swelling may help to improve the monitoring of the worsening of certain diseases. Thus, the quantification of ankle swelling and/or wrist swelling, as an example, may help to detect congestions in patients at home, in hospital environments or in other environments, at an early stage. Additionally or alternatively, the quantification may facilitate monitoring the improvement of treatments, such as medical treatments, e.g. by correlating the quantification to the treatment and/or by determining trends. Further, the quantification may assist healthcare professionals in decision-making, such as in making decisions regarding the discharge of the patient from hospitalization.

The diagnostic device as proposed herein may be designed in a robust and simple fashion. Thus, the diagnostic device may be fully or partially be designed as a bracelet that may be worn by the patient on the ankle and/or the wrist. Mechanically robust designs may be chosen, e.g. by designing the bracelet to comprise a linear, thin and flexible material that contains a functional part at one of its ends which contains the electromechanical actuator, e.g. a motor, wherein the other end of the bracelet may move along and/or through the electromechanical actuator, thereby increasing and/or reducing the circumference of a loop formed by the bracelet. When the electromechanical actuator, e.g. the motor, is actuated, e.g. rotated, the circumference of the bracelet is modified and, thus, the diameter and/or the equivalent diameter of the bracelet is also modified. Alternatively, the diagnostic device may be fully or partially designed as or integrated into a wearable device like a glove, a sock or a belt that can be worn by the patient.

The diagnostic device may be configured for reading, as an example, at least the rotation of the electromechanical actuator, e.g. the motor, to provide for a first coordinate or measurement variable, which may be part of the at least one item of circumference information and/or from which the at least one item of circumference information may be derived. As an example, this item of circumference information simply may be a distance x about which the bracelet is moved by the electromechanical actuator, the e.g. by reducing or increasing the circumference of the bracelet by the distance x. Further, the diagnostic device may be configured for reading the electrical power applied to the electromechanical actuator, e.g. the electrical power applied to the motor. This item of information may provide for a second coordinate or measurement variable, which may form the item of information on the electrical power applied to the electromechanical actuator and/or from which the item of information on the electrical power applied to the electromechanical actuator may be derived.

When the bracelet is worn on an ankle, a wrist or another body part, the measurement of the circumference and elasticity could be taken in predetermined, in determinable or even in irregular time intervals, such as every 5, 10, 15 minutes, every hour or the like. Thus, generally, as outlined above, the method or a part thereof, such as a measurement routine comprising steps iii. and iv. and optionally also step v., may fully or partially be performed repeatedly.

As outlined above, the method may comprise one or more measurement routines. As an example, a typical measurement routine may comprise the following steps to be performed when the bracelet is worn and loosely wound around the body part. As an example, the bracelet may be worn such that its circumference is sufficiently large to add a finger between the bracelet and the body part. This may refer to an initial configuration in which the circumference on the loop of the bracelet may have an initial absolute value and the length of the free end may have an initial absolute value. The measurement routine may be controlled by the control device. The measurement routine may comprise a routine start in which the electromechanical actuator is actuated, e.g. by rotating the motor in a predetermined direction known to decrease the circumference of the bracelet. During this step, the at least one item of information on the electrical power applied to the electromechanical actuator and the at least one item of circumference information are determined, such as by measuring electrical power and rotation and/or distance of the movement. The movement may be stopped at a predetermined point in time and/or under predetermined conditions, e.g. when a defined amount of electrical power applied to the electromechanical actuator is reached and/or after a predetermined distance. The routine may be controlled to comply with one or more predetermined safety conditions, such as with a safety limit regarding the amount of power applied to the electromechanical actuator and/or with a safety limit regarding a minimum circumference of the bracelet. The electromechanical actuator may then be controlled to move into an opposite direction, thereby increasing the circumference of the bracelet, e.g. by an appropriate rotation into an opposite direction, e.g. in order to recover the original configuration, state or position of the electromechanical actuator and/or the bracelet. This routine or sequence of action may be repeated. By evaluating the at least one item of information on the electrical power applied to the electromechanical actuator and the at least one item of circumference information, the at least one item of information on a status of the body tissue may be derived, e.g. by deriving at least one item of elasticity information and/or by deriving at least one item of body part circumference information of the body part, e.g. the ankle and/or wrist circumference and/or the elasticity of the ankle and/or wrist.

Summarizing and without excluding further possible embodiments, the following embodiments may be envisaged:

Embodiment 1: A diagnostic device for monitoring at least one body tissue of a patient, specifically for monitoring body swellings of a body tissue of the patient, the diagnostic device comprising:
a. at least one bracelet configured to be strapped around a body part of the patient, specifically around one or more of an ankle, a thigh, a lower thigh, a wrist, a forearm and an upper arm of the patient;
b. at least one electromechanical actuator configured for actively varying a circumference of the bracelet;
c. at least one measurement unit configured for determining at least one item of information on an electrical power applied to the electromechanical actuator and at least one item of circumference information on the circumference of the bracelet; and
d. at least one evaluation unit configured for determining at least one item of information on a status of the body tissue from the item of information on the electrical power applied to the electromechanical actuator and the item of circumference information.

Embodiment 2: The diagnostic device according to the preceding embodiment, wherein the evaluation unit is configured for determining a point of contact at which the circumference of the bracelet corresponds to the circumference of the body part.

Embodiment 3: The diagnostic device according to any one of the preceding embodiments, wherein the electromechanical actuator comprises at least one electrical motor.

Embodiment 4: The diagnostic device according to the preceding embodiment, wherein the electrical motor is selected from the group consisting of: a DC motor; a stepper motor.

Embodiment 5: The diagnostic device according to any one of the two preceding embodiments, wherein the electrical motor is configured for moving at least one first portion of the bracelet relative to at least one second portion of the bracelet, in order to vary the circumference of the bracelet.

Embodiment 6: The diagnostic device according to any one of the preceding embodiments, wherein the diagnostic device comprises at least one control unit configured for controlling at least one measurement routine of the diagnostic device.

Embodiment 7: The diagnostic device according to the preceding embodiment, wherein the measurement routine comprises at least one step of elasticity measurement, the step of elasticity measurement comprising:
adjusting the circumference of the bracelet to at least one first circumference,
varying the circumference of the bracelet from the first circumference to at least one second circumference, specifically at least one second circumference being smaller than the first circumference, wherein the item of information on the electrical power applied to the electromechanical actuator and the item of circumference information are recorded.

Embodiment 8: The diagnostic device according to the preceding embodiment, wherein the item of information on the status of the body tissue comprises at least one item of elasticity information on the body tissue, wherein the evaluation unit is configured for deriving the item of elasticity information from the item of information on the electrical power applied to the electromechanical actuator at the item of circumference information recorded during the step of elasticity measurement.

Embodiment 9: The diagnostic device according to the preceding embodiment, wherein the evaluation unit is configured for deriving the item of elasticity information from at least one of: a slope of a measurement curve indicating the electrical power applied to the electromechanical actuator and the circumference of the bracelet; a slope of a measurement curve indicating the circumference of the bracelet and the electrical power applied to the electromechanical actuator; at least two points of measurement, each point of measurement indicating the circumference of the bracelet and the electrical power applied to the electromechanical actuator.

Embodiment 10: The diagnostic device according to any one of the four preceding embodiments, wherein the measurement routine comprises at least one step of body part circumference measurement, the step of body part circumference measurement comprising:
decreasing the circumference of the bracelet, wherein the item of information on the electrical power applied to the electromechanical actuator and the item of circumference information are recorded.

Embodiment 11: The diagnostic device according to the preceding embodiment, wherein the item of information on the status of the body tissue comprises at least one item of body part circumference information on the body tissue, wherein the evaluation unit is configured for deriving the item of body part circumference information from the item of information on the electrical power applied to the electromechanical actuator and the item of circumference information recorded during the step of body part circumference measurement.

Embodiment 12: The diagnostic device according to the preceding embodiments, wherein the item of body part circumference information on the body tissue comprises a value associated with an absolute circumference of the body part the bracelet is to be strapped around.

Embodiment 13: The diagnostic device according to any one of the two preceding embodiments, wherein the evaluation unit is configured for determining the item of body part circumference information on the body tissue from at least one of: a slope of a measurement curve indicating the electrical power applied to the electromechanical actuator and the circumference of the bracelet; a slope of a measurement curve indicating the circumference of the bracelet and the electrical power applied to the electromechanical actuator, at least two points of measurement, each point of measurement indicating the circumference of the bracelet and the electrical power applied to the electromechanical actuator.

Embodiment 14: The diagnostic device according to any one of the four preceding embodiments, wherein the absolute value of the circumference of the bracelet is determined during the step of body part circumference measurement, particularly for determining the absolute circumference of the body part the bracelet is to be strapped around.

Embodiment 15: The diagnostic device according to the preceding embodiment, wherein the absolute value of the circumference of the bracelet is determined by at least one of:
considering an absolute length of the bracelet and an absolute length of a loose end of the bracelet, particularly wherein the absolute length of the entire bracelet is known and the absolute length of the loose end is determined by using a length sensor, more particularly wherein the length sensor is the electromechanical actuator; or
considering at least one position marker on the bracelet.

Embodiment 16: The diagnostic device according to any one of the preceding embodiments, wherein the bracelet comprises at least one toothed belt or rack configured for interacting with the electromechanical actuator.

Embodiment 17: The diagnostic device according to the preceding embodiment, wherein the diagnostic device comprises at least one pinion driven by the electromechanical actuator, the pinion being configured for interacting with the toothed belt or rack.

Embodiment 18: The diagnostic device according to any one of the preceding embodiments, wherein the at least one measurement unit comprises at least one electrical measurement device configured for determining the at least one item of information on the electrical power applied to the electromechanical actuator and at least one position measurement device configured for determining the at least one item of circumference information on the circumference of the bracelet.

Embodiment 19: The diagnostic device according to the preceding embodiment, wherein the electrical measurement device comprises at least one of a voltage measurement device and a current measurement device.

Embodiment 20: The diagnostic device according to any one of the two preceding embodiments, wherein the position measurement device is configured for determining at least one of: an absolute position of at least one part of the bracelet; a relative position of at least one first portion of the bracelet relative to at least one second portion of the bracelet; a temporal change of an absolute position of at least one part of the bracelet; or a change in a relative position of at least one first portion of the bracelet relative to at least one second portion of the bracelet.

Embodiment 21: A method of monitoring at least one body tissue of a patient, the method comprising:
i. providing at least one diagnostic device according to any one of the preceding embodiments;
ii. strapping the bracelet around a body part of the patient;
iii. actively varying the circumference of the bracelet by using the electromechanical actuator;
iv. determining at least one item of information on an electrical power applied to the electromechanical actuator and at least one item of circumference information on the circumference of the bracelet by using the measurement unit; and
v. determining at least one item of information on a status of the body tissue from the item of information on the electrical power applied to the electromechanical actuator and the item of circumference information by using the at least one evaluation unit.

Embodiment 22: The method according to the preceding embodiment, wherein step v. further comprises determining a point of contact at which the circumference of the bracelet corresponds to the circumference of the body part by using the at least one evaluation unit. Embodiment 23: The method according to any one of the two preceding embodiments, wherein at least step v. is computer-implemented.

Embodiment 24: A computer program comprising instructions which, when the program is executed by the evaluation unit of the diagnostic device according to any one of the preceding embodiments referring to a diagnostic device, cause the evaluation unit to perform step v. of the method according to any one of the preceding embodiments referring to a method.

Embodiment 25: A computer-readable storage medium, specifically a non-volatile computer-readable storage medium, comprising instructions which, when the instructions are executed by the evaluation unit of the diagnostic device according to any one of the preceding embodiments referring to a diagnostic device, cause the evaluation unit to perform step v. of the method according to any one of the preceding embodiments referring to a method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the figures:

FIG. 1 shows an embodiment of a diagnostic device in a cross-sectional view, with a bracelet in an open state;

FIG. 2 shows the diagnostic device of FIG. 1 with the bracelet in a closed state;

FIG. 3 shows an interaction of a pinion and the electromechanical actuator of the diagnostic device of FIG. 2 with a toothed belt of the bracelet;

FIGS. 4A and 4B show a measurement routine including a narrowing of the circumference of the bracelet of FIG. 2 when in a state with the bracelet being wound around a body part of a patient; and

FIG. 5 shows the determination of at least one item of information on a status of the body tissue from a measurement routine as shown e.g. in FIGS. 4A and 4B.

### DETAILED DESCRIPTION OF THE EMBODIMENTS

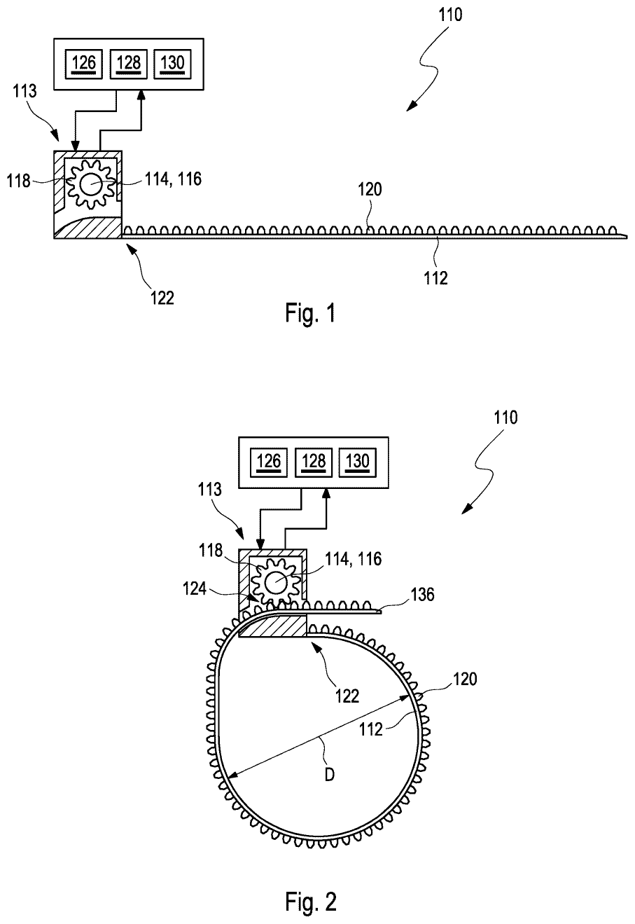

In FIGS. 1 and 2, an embodiment of a diagnostic device 110 for monitoring at least one body tissue of a patient is shown in a cross-sectional view in different configurations. The diagnostic device 110, in the embodiments shown, comprises a bracelet 112 configured to be strapped around a body part of the patient. In FIG. 1, the bracelet 112 is shown in an open configuration, and in FIG. 2 the bracelet 112 is shown in a closed configuration.

The diagnostic device 110 further comprises at least one electromechanical actuator 114. In the embodiment shown, the electromechanical actuator 114, specifically, may comprise an electric motor 116. The diagnostic device 110, as also shown in the embodiment, may further comprise at least one pinion 118 coupled to the electromechanical actuator 114. The pinion 118 may form a gear interacting with the bracelet 112. As an example, the pinion 118 may interact with a toothed belt 120 coupled to the bracelet 112 and/or integrated into the bracelet 112, wherein the toothed belt 120 may extend along the full length of the bracelet 112 or along a part of the length of the bracelet 112.

The electromechanical actuator 114 may be coupled or fixed to a first portion 122 of the bracelet 112, e.g. to an end portion. As can be seen when comparing FIGS. 1 and 2, in the closed configuration, the bracelet 112 may form a loop having a diameter D, which, in case the loop is a circular loop, corresponds to the circumference of the loop divided by π. In case of a noncircular loop, the diameter D may be determined as an equivalent diameter, being the diameter of a circle having the same cross-sectional area as the noncircular loop. The diameter D of the loop may be proportional to the circumference of the loop.

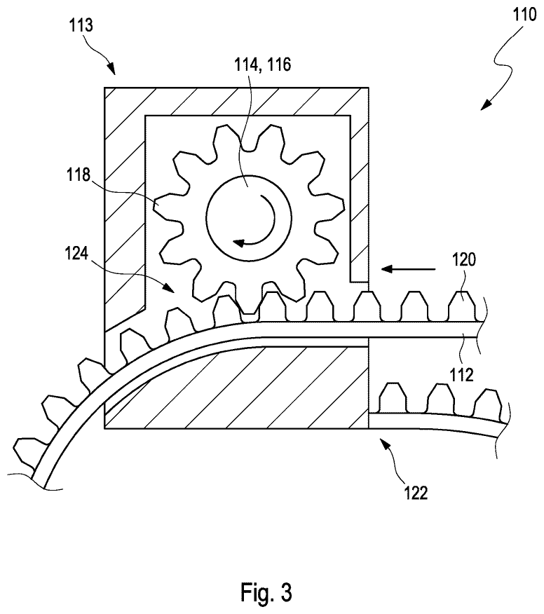

As further can be seen in FIG. 2, the bracelet 110 may comprise a fastener 113 designed for forming the loop of the bracelet 110, wherein the electromechanical actuator 114 is or is comprised by the fastener 113. The fastener 113 may refer to a unit, particularly a unit separate from a band used for forming the bracelet 113, which connects the ends of the band together such that a loop is formed. The fastener may 113 may be opened and/or closed. The fastener 113 may be a clasp and/or a closure, particularly required for forming the loop of the bracelet 110. The electromechanical actuator 114 may interact with the bracelet 112 in such a way that a second portion 124 of the bracelet 112 is moved relative to the first portion 122. This is shown in detail in FIG. 3, which shows an enlarged view of the electromechanical actuator 114 and the interaction of the pinion 118 with the toothed belt 120 of the bracelet 112. By rotating the pinion 118, e.g. in a clockwise direction in FIG. 3, the second portion 124 of the bracelet 112 is moved, e.g. to the left in FIG. 3. Thereby, the circumference and the diameter D of the loop in FIG. 2 may be changed. As an example, in the direction of movement shown in FIG. 3 in conjunction with the setup of FIG. 2, the diameter D and thereby also the circumference is increased. By reversing the direction of movement of the pinion 118 to a counterclockwise direction, the diameter D and thereby also the circumference of may be reduced.

For monitoring the body tissue of the patient and for generating at least one item of information on a status of the body tissue, the diagnostic device 110 comprises further elements, which are schematically shown in FIGS. 1 and 2. Thus, firstly, the diagnostic device 110 comprises at least one measurement unit 126 configured for determining at least one item of information on an electrical power applied to the electromechanical actuator 114 and at least one item of circumference information on the circumference of the bracelet. Further, the diagnostic device 110 comprises at least one evaluation unit 128 configured for determining at least one item of information on the status of the body tissue from the item of information on the electrical power applied to the electromechanical actuator and the item of circumference information. Further, optionally, the diagnostic device 110 may comprise at least one control unit 130 configured for controlling the diagnostic device 110, e.g. for controlling at least one measurement routine of the diagnostic device 110. Even though depicted as single and separate components in the Figures, the units 126, 128 and 130, each, may also fully or partially be integrated into one or more other components, such as into the electromechanical actuator 114 and/or into each other. The units 126, 128 and 130 may be separate from the bracelet 112 may be connected to the electromechanical actuator 114 in a wireless or wire bound fashion or, alternatively, may also fully or partially be integrated into the bracelet 112 and/or the electromechanical actuator 114.

As an example, the measurement unit 126 may fully or partially be coupled to an electrical energy supply or power supply of the electromechanical actuator 114, which may be an internal and/or external power supply. Further, the measurement unit 126 may derive the at least one item of circumference information from a relative position of the first and second portions 122, 124 and/or from a status of the electromechanical actuator 114, e.g. from a rotational position of the pinion 118. Various concepts are feasible and may be implemented in the present invention.

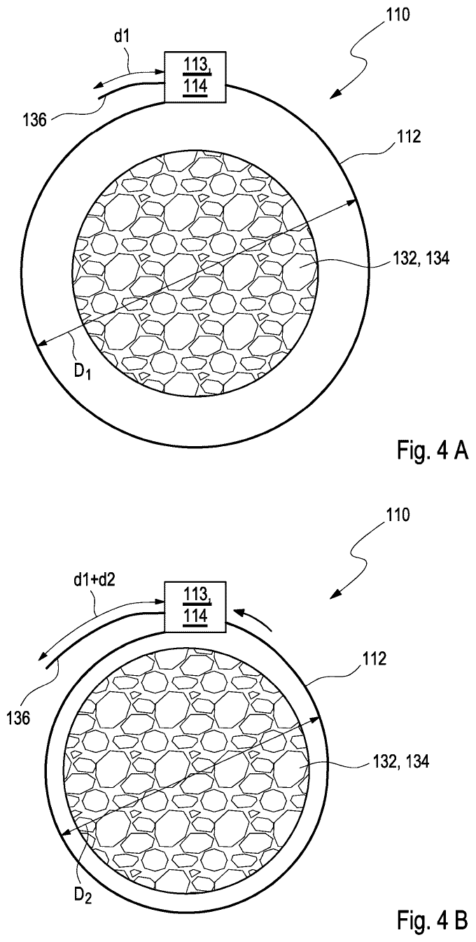

In FIGS. 4A and 4B, the diagnostic device 110 is shown with the bracelet 112 being wound around a body part 132 of a patient, the body part 132 having body tissue 134. The sequence of these Figures may also provide for a measurement routine and/or may be part of a measurement routine, e.g. controlled by the control unit 130. Thus, firstly, the bracelet 112, in FIG. 4A, is wound loosely around the body part 132, e.g. an ankle and/or a wrist of the patient. A loose end 136 of the bracelet 112, protruding from the electromechanical actuator 114, may have a length d₁. Thereby, the absolute circumference of the loop formed by the bracelet 112 may be determined by considering the absolute length of the bracelet 112 and the absolute length of the loose end 136. For doing so, the absolute length d of the entire bracelet 112, particularly of a band of the bracelet 112, may be predetermined or known. In addition, the length d₁ of the loose end 136 may be predetermined, known or determined by using a length sensor. The length sensor may be the electromechanical actuator 114. The loop of the bracelet 112 may have an initial circumference, particularly when the bracelet 112 is just applied to the body part 132 of the patient. As an example, the electromechanical actuator 114 may count the number of teeth of the toothed belt 120 that were inserted into the electromechanical actuator 114 during the application of the bracelet 112 to the body part 132 of the patient for determining the initial absolute length d₁ of the loose end 136. The absolute length of the initial circumference may be derived by considering, particularly subtracting, the initial absolute length d₁ of the loose end 136 from the known absolute length d of the entire bracelet 112. Alternatively, position markers on the bracelet 112 may be used for determining the absolute circumference of the bracelet 112, particularly of the loop of the bracelet.

By moving the electromechanical actuator 114, e.g. by rotating the motor 116, the diameter D of the bracelet is changed, from D₁ in FIG. 4A to D₂ in FIG. 4B. Thereby the length of the loose end 136 is increased from a length d₁ in FIG. 4A to a length d₁+d₂ in FIG. 4B. As an example, in case of a circular loop the diameter is reduced from D₁=(d−d₁)/π in FIG. 4A, with d being the length of the entire bracelet 112, to D₂=(d−d₁−d₂)/π in FIG. 4B.

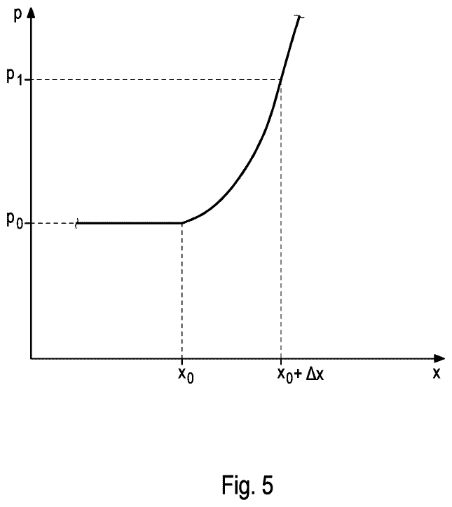

This measurement routine can be used for deriving the one or more items of information on the status of the body tissue 134. An example is shown in FIG. 5, showing a graph of the electrical power p applied to the electromechanical actuator 114 on the vertical axis, as a function of the position of the first portion 122 with respect to the second portion 124, denoted by x. In principle, any type of information may be used for providing circumference information, including a position of the pinion 118, a longitudinal coordinate along the bracelet 112 or the like.

As can be seen in FIG. 5, when the electromechanical actuator 114 is actuated, firstly, a low power po has to be applied for varying the circumference of the bracelet 112, as long as the bracelet 112 is loosely wound around the body part 132. Once the circumference of the bracelet 112, however, fits with the circumference of the body part 132, equivalent approximately to the situation in FIG. 4B, the body tissue 134 starts exerting a counterforce to the further narrowing of the bracelet 112. Therefore, the electrical power p which has to be provided to the electromechanical actuator 114 for further decreasing the circumference of the bracelet 112 starts rising. This is the case at position x₀ in FIG. 5. The rise may be detected, e.g. by the measurement unit and/or by the evaluation unit. Since there is a direct relationship between position x₀ and the circumference of the bracelet 112, which may be determined e.g. by geometrical considerations and/or by calibration measurements, x₀ may provide for an body part circumference information on the body tissue 134, e.g. a circumference of the body part 132.

When further moving on, the counterforce rises, e.g. in accordance with Hooke's law or other physical relationships between a force to be applied in an extension or compression of an object, particularly of a body tissue. As an example, when further increasing x by a distance Δx, the electrical power p to be applied to the electromechanical actuator 114 may rise from p₀ to p₁. Thereby, a slope may be determined, e.g. by dividing the rise in power by the change in distance: S=Δp/Δx=(p₁−p₀)/Δx. The slope may provide for an item of elasticity information.

By combining the above-mentioned options, as an example, two different parameters being of interest for determining the status of the body tissue 134 may be derived. Thus, the at least one item of body part circumference information and the at least one item of elasticity information may be generated. Instead of the variables shown above, other variables may be used, such as forces applied by the electromechanical actuator 114 and/or angular information. Various options are feasible.

### LIST OF REFERENCE NUMBERS

110 Diagnostic device
112 Bracelet
113 Fastener
114 Electromechanical actuator
116 Motor
118 Pinion
120 Toothed belt
122 First portion
124 Second portion
126 Measurement unit
128 Evaluation unit
130 Control unit
132 Body part
134 Body tissue
136 Loose end

1. A diagnostic device for monitoring at least one body tissue of a patient, the diagnostic device comprising:
a. at least one bracelet configured to be strapped around a body part of the patient;
b. at least one electromechanical actuator configured for actively varying a circumference of the bracelet
c. at least one measurement unit configured for determining at least one item of information on an electrical power applied to the electromechanical actuator and at least one item of circumference information on the circumference of the bracelet; and
d. at least one evaluation unit configured for determining at least one item of information on a status of the body tissue from the item of information on the electrical power applied to the electromechanical actuator and the item of circumference information, wherein the diagnostic device comprises at least one control unit configured for controlling at least one measurement routine of the diagnostic device, wherein the measurement routine comprises at least one step of elasticity measurement, the step of elasticity measurement comprising:
adjusting the circumference of the bracelet to at least one first circumference,
varying the circumference of the bracelet from the first circumference to at least one second circumference, wherein the item of information on the electrical power applied to the electromechanical actuator and the item of circumference information are recorded.
wherein the item of information on the status of the body tissue comprises at least one item of elasticity information on the body tissue, wherein the evaluation unit is configured for deriving the item of elasticity information from the item of information on the electrical power applied to the electromechanical actuator at the item of circumference information recorded during the step of elasticity measurement, wherein the evaluation unit is configured for deriving the item of elasticity information from at least one of: a slope of a measurement curve indicating the electrical power applied to the electromechanical actuator and the circumference of the bracelet; a slope of a measurement curve indicating the circumference of the bracelet and the electrical power applied to the electromechanical actuator,
wherein the evaluation unit is configured for determining a point of contact at which the circumference of the bracelet corresponds to the circumference of the body part, wherein by, considering the point of contact, the absolute value of the circumference of the body part is determined, wherein the point of contact is determined by detecting the onset of a rise in electrical power required to be applied to the actuator by monitoring the slope of a curve indicating the electrical power as a function of the circumference information,
wherein the at least one item of information on a status of the body tissue comprises the at least one item of elasticity information and the at least one item of body part circumference information of the body part, wherein the item of body part circumference information provides information on the absolute circumference of the body part.

2. The diagnostic device according to claim 1, wherein the electromechanical actuator comprises at least one electrical motor.

3. The diagnostic device according to claim 2, wherein the electrical motor is selected from the group consisting of: a DC motor; a stepper motor.

4. The diagnostic device according to claim 2, wherein the electrical motor is configured for moving at least one first portion of the bracelet relative to at least one second portion of the bracelet in order to vary the circumference of the bracelet

5. (canceled)

6. (canceled)

7. (canceled)

8. (canceled)

9. The diagnostic device according to claim 1, wherein the measurement routine comprises at least one step of body part circumference measurement, the step of body part circumference measurement comprising:
decreasing the circumference of the bracelet, wherein the item of information on the electrical power applied to the electromechanical actuator and the item of circumference information are recorded.

10. The diagnostic device according to claim 1, wherein the item of information on the status of the body tissue comprises at least one item of body part circumference information on the body tissue wherein the evaluation unit is configured for deriving the item of body part circumference information from the item of information on the electrical power applied to the electromechanical actuator and the item of circumference information recorded during the step of body part circumference measurement.

11. The diagnostic device according to claim 10, wherein the item of body part circumference information on the body tissue comprises a value associated with an absolute circumference of the body part the bracelet is to be strapped around.

12. The diagnostic device according to claim 10, wherein the evaluation unit is configured for determining the item of body part circumference information on the body tissue from at least one of: a slope of a measurement curve indicating the electrical power applied to the electromechanical actuator and the circumference of the bracelet; a slope of a measurement curve indicating the circumference of the bracelet and the electrical power applied to the electromechanical actuator; at least two points of measurement, each point of measurement indicating the circumference of the bracelet and the electrical power applied to the electromechanical actuator.

13. The diagnostic device according to claim 1, wherein the bracelet comprises at least one toothed belt configured for interacting with the electromechanical actuator.

14. The diagnostic device according to claim 1, wherein the diagnostic device comprises at least one pinion driven by the electromechanical actuator, the pinion being configured for interacting with the toothed belt.

15. The diagnostic device according to claim 1, wherein the at least one measurement unit comprises at least one electrical measurement device configured for determining the at least one item of information on the electrical power applied to the electromechanical actuator and at least one position measurement device configured for determining the at least one item of circumference information on the circumference of the bracelet.

16. A method of monitoring at least one body tissue of a patient, the method comprising:
i. providing at least one diagnostic device according to claim 1;
ii. strapping the bracelet around a body part of the patient;
iii. actively varying the circumference of the bracelet by using the electromechanical actuator;
iv. determining at least one item of information on an electrical power applied to the electromechanical actuator and at least one item of circumference information on the circumference of the bracelet by using the measurement unit and
v. determining at least one item of information on a status of the body tissue from the item of information on the electrical power applied to the electromechanical actuator and the item of circumference information by using the at least one evaluation unit, and determining a point of contact at which the circumference of the bracelet corresponds to the circumference of the body part by using the at least one evaluation unit.
